(19) United States
(12) Patent Application Publication
(10) Pub. No.: US 2023/0384283 A1
(43) Pub. Date: Nov. 30, 2023

(54) Method, system, computer equipment and storage medium for nitrogen pollution hierarchical calculation in water environment

(71) Applicant: Institute of Eco-environmental and Soil Sciences, Guangdong Academy of Sciences, Guangzhou (CN)

(72) Inventors: Bin HE, Guangzhou (CN); Qixiu LI, Guangzhou (CN)

(21) Appl. No.: 18/321,846

(22) Filed: May 23, 2023

(30) Foreign Application Priority Data
May 25, 2022 (CN) 2022105727981

(51) Int. Cl. G01N 33/24; G01N 33/00

(52) U.S. Cl. CPC G01N 33/24; G01N 33/0098

(57) ABSTRACT

Disclosed are a method, a system, a computer equipment and a storage medium for nitrogen pollution hierarchical calculation in a water environment, and the method includes the following steps: carrying out a gridding processing on a watershed to obtain watershed grids; constructing a nitrogen pollution biogeochemical circulation model according to calculation methods and interrelations of carbon and nitrogen variables; establishing the nitrogen pollution biogeochemical circulation model in each of the watershed grids; and calculating a total non-point source nitrogen pollution in the watershed according to the nitrogen pollution biogeochemical circulation model in each of the watershed grids.

### CROSS-REFERENCE TO RELATED APPLICATIONS

This application claims priority to Chinese Patent Application No. 202210572798.1, filed on May 25, 2022, the contents of which are hereby incorporated by reference.

### TECHNICAL FIELD

The application relates to a method, a system, a computer equipment and a storage medium for nitrogen pollution hierarchical calculation in a water environment, belonging to the field of non-point source nitrogen pollution calculation in water environment nitrogen pollution.

### BACKGROUND

At present, agricultural non-point source nitrogen pollution accounts for a large proportion of water pollution, and it becomes increasingly serious. However, the treatment of agricultural non-point source nitrogen pollution requires quantitative research on nitrogen pollutants, so the quantitative research on nitrogen pollutants has become an urgent problem to be solved. In order to quantitatively study non-point source nitrogen pollution, it is generally necessary to establish corresponding models. In the actual quantitative research process, the models of non-point source nitrogen pollution may be classified into two kinds: the statistical model based on output coefficients and the distributive hydrological and water quality model.

The statistical model based on the output coefficient method is to induce and count the measured values of nitrogen concentration and flow data of rivers in the watershed according to a certain method, and adjust the output coefficient according to experience, so as to obtain the nitrogen pollution load to represent the nitrogen pollution degree of the whole region. However, the parameters used in the statistical model based on output coefficients are designed according to experience, without considering different accuracy levels of different model parameters in calculating nitrogen pollution loads, or the physical significance of nitrogen cycle within the watershed.

The distributed hydrological and water quality model is to use the model to calculate the water quality and flow of each grid in the watershed, and then calculate the nitrogen load that finally flows to the outlet of the watershed according to the river transport model to represent the nitrogen pollution degree of the whole region. However, in the calculation of nitrogen load in the watershed, the cycle process of nitrogen element is not considered, and the calculation result of nitrogen pollution load is single, and only a single result of nitrogen pollution load at the estuary of the watershed or in the grid of the watershed is provided, and the comprehensive spatial and temporal distribution of nitrogen pollution components in the watershed in physics, chemistry and biology is not revealed, which is not conducive to evaluating the comprehensive source and spatial and temporal distribution of nitrogen pollution.

### SUMMARY

In view of this, the application provides a method, a system, a computer equipment and a storage medium for nitrogen pollution hierarchical calculation in a water environment, considers the influence of land use, soil, topography and other factors on grid nitrogen pollution calculation, clarifies the physical meaning of different model parameters, and makes the determination of each parameter more scientific and reasonable; meanwhile, aiming at the watershed scale nitrogen pollution, the nitrogen pollution biogeochemical circulation model established based on the grid is established, which considers the cycle process and mechanism of nitrogen pollution in physics, chemistry and biology, and may comprehensively reflect the spatial and temporal distribution of pollutant concentrations of nitrogen pollution in river watersheds and water bodies, thus making the evaluation results more complete.

The first objective of the application is to provide a method for nitrogen pollution hierarchical calculation in a water environment.

The second objective of the present application is to provide a system for nitrogen pollution hierarchical calculation in a water environment.

The third objective of the present application is to provide a computer equipment.

The fourth objective of the present application is to provide a storage medium.

The first objective of the present application may be achieved by adopting the following technical scheme.

A method for nitrogen pollution hierarchical calculation in a water environment, including following steps:

S1, carrying out a gridding processing on a watershed to obtain watershed grids;

S2, constructing a nitrogen pollution biogeochemical circulation model according to calculation methods and interrelations of carbon and nitrogen variables;

S3, establishing the nitrogen pollution biogeochemical circulation model in each of the watershed grids; and

S4, calculating a total non-point source nitrogen pollution in the watershed according to the nitrogen pollution biogeochemical circulation model in each of the watershed grids.

Further, constructing a nitrogen pollution biogeochemical circulation model according to calculation methods and interrelations of carbon and nitrogen variables specifically comprises as follows: determining a variable C_{VEG} of an organic carbon element content in plants, a variable C_{VEG} of an organic nitrogen element content in the plants, a variable C_{DET} of an organic carbon content in a surface soil, a variable N_{DET} of an organic nitrogen content in the surface soil, a variable C_{HUM} of the organic carbon content in a humus soil and a variable N_{HUM} the organic nitrogen content in the humus soil; and

determining the calculation methods and the interrelations of C_{VEG}, N_{VEG} , C_{DET}, N_{DET}, C_{HUM} and N_{HUM}, and completing a construction of the nitrogen pollution biogeochemical circulation model according to a nitrogen pollution load calculation model.

Further, the calculation methods of C_{VEG}, N_{VEG} , C_{DET}, N_{DET}, C_{HUM} and N_{HUM} are as follows:

a calculation method of the organic carbon content in the plants:

$$\frac{\partial {C}_{VEG}}{\partial t} = gpp - {C}_{trr} - {C}_{f} ;$$

a calculation method of the organic carbon content in the surface soil:

$$\frac{\partial {C}_{DET}}{\partial t} = {C}_{f} - {C}_{dr} - {C}_{dh} ;$$

a calculation method of the organic carbon content in the humus soil:

$$\frac{\partial {C}_{HUM}}{\partial t} = {C}_{dh} - {C}_{hr} - {C}_{hcar} ;$$

a calculation method of the organic nitrogen content in the plants:

$$\frac{\partial {N}_{VEG}}{\partial t} = {N}_{uptake} - {N}_{f} + {N}_{fix} ;$$

a calculation method of the organic nitrogen content in the surface soil:

$$\frac{\partial {N}_{DET}}{\partial t} = {N}_{f} - {N}_{mind} - {N}_{dh} ;$$

a calculation method of the organic nitrogen content in the humus soil:

$$\frac{\partial {N}_{HUM}}{\partial t} = {N}_{dh} - {N}_{min h} ;$$

where wherein gpp stands for a flux of photosynthesis as in gross primary production, Cₜᵣᵣ stands for a flux of respiration of trunk and root, C_{f} stands for a flux of litter-fall from leaf, trunk, and root as in carbon, C_{dr} stands for a flux of detritus decomposition as in carbon, C_{dh} stands for a flux of detritus humification as in carbon; Cₕᵣ stands for a flux of humus decomposition as in carbon, Cₕₑₐᵣ stands for a flux of humus carbonization as in carbon, Nᵤₚₜₐₖₑ stands for a flux of nitrogen uptake by plant, N_{f} stands for a flux of litter-fall from leaf, trunk, and root as in nitrogen, N_{fix} stands for a flux of nitrogen fixation as in nitrogen, N_{mind} stands for a flux of mineralization of the organic nitrogen, N_{dh} stands for a flux of detritus humification as in nitrogen, and N_{min h} stands for a inorganic nitridation of the humus soil.

Further, the nitrogen pollution load calculation model includes a calculation of nitrogen pollution settlement from precipitation sources, a calculation of nitrogen absorption by crops, a calculation of nitrate nitrogen deoxidation process and a calculation of nitrate nitrogen leaching.

Further, the nitrogen pollution from precipitation sources includes ammonia nitrogen element by a precipitation settlement and nitrate nitrogen element by the precipitation settlement; and a calculation formula of the ammonia nitrogen element by the precipitation settlement is as follows:

depo_{AMM}=⅓×C_{N}×N_{PRE}

The calculation formula of the nitrate nitrogen element by the precipitation settlement is as follows:

depo_{NIT}=⅔×C_{N}×N_{PRE};

where depo_{AMM} stands for the ammonia nitrogen in an atmospheric settlement, depo_{NIT} stands for the nitrate nitrogen in the atmospheric settlement, C_{N} stands for a nitrogen settlement coefficient, and N_{PRE} stands for the nitrogen element in the precipitation.

Further, specific formulae for calculating the nitrogen absorption by crops are as follows:

$${N}_{uptake} = ( \frac{{N}_{max} {K}_{s , update} ( {N}_{AMM} + {N}_{NIT} )}{{K}_{uptake} + {K}_{s , uptake} ( {N}_{AMM} + {N}_{NIT} )} ) \times {Q}_{10 , {N}_{uptake}}^{\frac{{T}_{S} - {T}_{opt , uptake}}{10}} ; \\ {K}_{s , uptake} = 0.9 \times {SW}_{{I}^{3}} + 0.1 ;$$

where Nₘₐₓ represents a maximum nitrogen uptake of the crops, N_{AMM} represents an ammonia nitrogen content in the soil, N_{NIT} represents a nitrate nitrogen content in the soil, and SWI represents a soil moisture index.

Further, specific formulae for calculating the nitrate nitrogen deoxidation process and the calculation formula of the nitrate nitrogen leaching are as follows:

the calculation formula of the nitrate nitrogen deoxidation process is as follows:

$$denitr = {N}_{NIT} [ 1 - exp ( - 1.4 {f}_{deni , t} C ) ] {num}_{day} ; \\ {f}_{deni , t} = max [ \begin{matrix}0.1 \\ \frac{t}{t + exp ( 9.93 - 0.312 t )}\end{matrix} ] ;$$

where denitr stands for a denitrification of the nitrate nitrogen, N_{NIT} stands for the nitrate nitrogen content in the soil, and num_{day} stands for a number of days;

the calculation formula of the nitrate nitrogen leaching is as follows:

$$K ( SWI ) = {K}_{3} {SWI}^{3 + \frac{2}{λ}} ;$$

where Kₛ is a soil unsaturated permeability coefficient, SWI is the soil moisture index, and λ is a pore size distribution index; the formula for calculating the pore size distribution index is as follows:

$$λ = \frac{1}{b} ;$$

where b is an empirical constant.

The second objective of the present application may be achieved by adopting the following technical scheme:

a system for nitrogen pollution hierarchical calculation in a water environment, including:

a grid processing unit used for carrying out a gridding processing on a watershed to obtain watershed grids;

a construction unit used for constructing a nitrogen pollution biogeochemical circulation model according to calculation methods and interrelations of t carbon and nitrogen variables;

a grid model establishing unit for constructing the nitrogen pollution biogeochemical circulation model in each of the watershed grids; and

a calculation unit used for calculating the total non-point source nitrogen pollution in the watershed according to the nitrogen pollution biogeochemical circulation model in each of the watershed grids.

The third objective of the present application may be achieved by adopting the following technical scheme:

a computer equipment, including a processor and a memory for storing programs executable by the processor, and when the processor executes the programs stored in the memory, the method for nitrogen pollution hierarchical calculation in a water environment is realized.

The fourth objective of the present application may be achieved by adopting the following technical scheme:

a storage medium for storing programs, and when the programs are executed by the processor, the method for nitrogen pollution hierarchical calculation in a water environment is realized.

Compared with the prior art, the application has the following beneficial effects.

According to the application, based on the difference of each grid and considering the physical meaning of model parameters in different grids, the parameter setting in the grid is more scientific and reasonable; meanwhile, the nitrogen pollution biogeochemical circulation model established based on the grid takes into account the physical, chemical and biological cycle process and mechanism of nitrogen pollution, and may comprehensively reflect the spatial and temporal distribution of pollutant concentrations of various nitrogen components in nitrogen pollution in river watersheds and water bodies, so the evaluation results of non-point source nitrogen pollution in river watersheds and water environment are more reliable, scientific and comprehensive.

### BRIEF DESCRIPTION OF THE DRAWINGS

In order to explain the embodiments of the present application or the technical scheme in the prior art more clearly, the drawings needed to be used in the description of the embodiments or the prior art will be briefly introduced below. Obviously, the drawings in the following description are only some embodiments of the present application, and other drawings may be obtained according to the structures shown in these drawings without creative work for ordinary people in the field.

FIG. 1 is a flow chart of the method for nitrogen pollution hierarchical calculation in a water environment according to Embodiment 1 of the present application.

FIG. 2 is a structural diagram of the water environment nitrogen pollution biogeochemical circulation model in Embodiment 1 of the present application.

FIG. 3 is a flowchart of a system for nitrogen pollution hierarchical calculation in a water environment according to Embodiment 2 of the present application.

FIG. 4 is a structural block diagram of a computer equipment according to Embodiment 3 of the present application.

### DETAILED DESCRIPTION OF THE EMBODIMENTS

In order to make the objectives, technical schemes and advantages of the embodiments of the application clearer, the technical schemes in the embodiments of the application will be described clearly and completely with the attached drawings. Obviously, the embodiments are a part of embodiments of the application, but not the whole embodiments. Based on the embodiments of the application, all other embodiments obtained by ordinary technicians in the field without creative labor belong to the scope of protection of the application.

### Embodiment 1

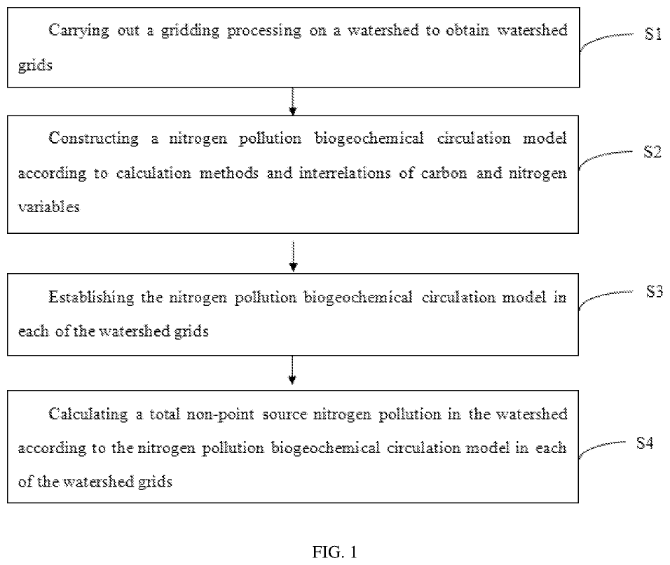

As shown in FIG. 1, this embodiment provides a method for nitrogen pollution hierarchical calculation in a water environment, including the following steps:

S101, a gridding processing is carried out on a watershed to obtain watershed grids;

S102, a nitrogen pollution biogeochemical circulation model is constructed according to calculation methods and interrelations of carbon and nitrogen variables.

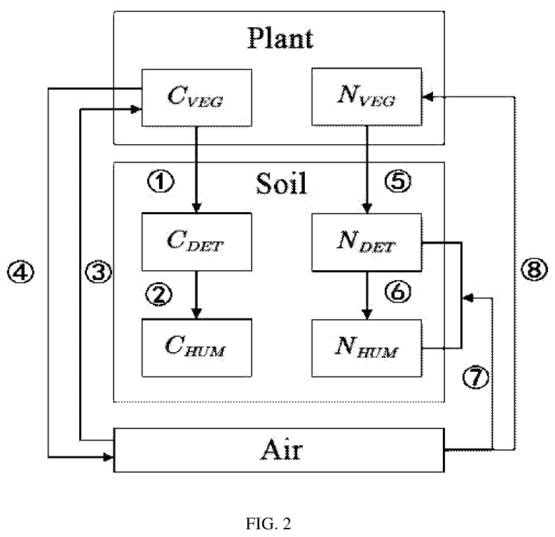

The model design is shown in FIG. 2, and the specific steps are as follows:

S1021, the key variables are determined in the biogeochemical circulation model of nitrogen pollution.

Determining a variable C_{VEG} of an organic carbon element content in plants, a variable N_{VEG} of an organic nitrogen element content in the plants, a variable C_{DET} of an organic carbon content in a surface soil, a variable N_{DET} of an organic nitrogen content in the surface soil, a variable C_{HUM} of the organic carbon content in a humus soil and a variable N_{HUM} of the organic nitrogen content in the humus soil;

S1022, the calculation methods and the interrelations of C_{VEG}, N_{VEG}, C_{DET}, N_{DET}, C_{HUM} and N_{HUM} are determined, and completing a construction of the nitrogen pollution biogeochemical circulation model according to a nitrogen pollution load calculation model.

The key variables are calculated as follows:

1) The calculation formula for determining the organic carbon content in the plants (crops) is as follows:

$$\frac{\partial {C}_{VEG}}{\partial t} = gpp - {C}_{trr} - {C}_{f} ; \qquad 3.1$$

Formula 3.1 shows that the organic carbon content in plants increases through photosynthesis, but decreases due to respiration and the falling of leaves, branches and roots.

2) Calculation method for determining organic carbon content in the surface soil:

$$\frac{\partial {C}_{DET}}{\partial t} = {C}_{f} - {C}_{dr} - {C}_{dh} ; \qquad 3.2$$

Formula 3.2 shows that the organic carbon content in the surface soil is increased by leaves, branches and roots falling to the surface, and it is decreased by decomposition of organic carbon on the surface and humification into the underground.

3) Calculation method for determining organic carbon content in the underground soil:

$$\frac{\partial {C}_{HUM}}{\partial t} = {C}_{dh} - {C}_{hr} - {C}_{hcar} ; \qquad 3.3$$

Formula 3.3 shows that the organic carbon content in underground soil increases with the humification of surface soil, and decreases with the decomposition and fibrosis of organic

4) Calculation method for determining the organic nitrogen content in the plants (crops):

$$\frac{\partial {N}_{VEG}}{\partial t} = {N}_{uptake} - {N}_{f} + {N}_{fix} ; \qquad 3.4$$

Formula 3.4 shows that the organic nitrogen content in the plants is increased by the absorption of nitrogen fertilizer by the plants (crops) and the biological nitrogen fixation activities of the plants (crops), but decreased by respiration and the fall of leaves, branches and roots.

5) Calculation method for determining the organic nitrogen content in the surface soil:

$$\frac{\partial {N}_{DET}}{\partial t} = {N}_{f} - {N}_{mind} - {N}_{dh} ; \qquad 3.5$$

Formula 3.5 shows that the organic nitrogen content in the surface soil is increased by leaves, branches and roots falling to the surface, and it is decreased by mineralization of the organic nitrogen on the surface and humification into the underground.

6) Calculation method for determining the organic nitrogen in the underground soil:

$$\frac{\partial {N}_{HUM}}{\partial t} = {N}_{dh} - {N}_{min h} ; \qquad 3.6$$

Formula 3.6 shows that the organic nitrogen content in the underground soil increases with the humification of surface soil and decreases with the inorganization of the organic nitrogen;

where, gpp stands for a flux of photosynthesis as in gross primary production, Cₜᵣᵣ stands for a flux of respiration of trunk and root, C_{f} stands for a flux of litter-fall from leaf, trunk, and root as in carbon, C_{dr} stands for a flux of detritus decomposition as in carbon, C_{dh} stands for a flux of detritus humification as in carbon; Cₕᵣ stands for a flux of humus decomposition as in carbon, C_{hcar} stands for a flux of humus carbonization as in carbon, Nᵤₚₜₐₖₑstands for a flux of nitrogen uptake by plant, N_{f} stands for a flux of litter-fall from leaf, trunk, and root as in nitrogen, N_{fix} stands for a flux of nitrogen fixation as in nitrogen, N_{mind} stands for a flux of mineralization of the organic nitrogen, N_{dh} stands for a flux of detritus humification as in nitrogen, and N_{min h} stands for a inorganic nitridation of the humus soil.

The interrelation of the key variables is as follows:

The model design is shown in FIG. 2, and the numbers \textcircled{1}-\textcircled{8} represent the important interaction among the variables: \textcircled{1} (C_{f}) represents that the organic carbon distributed in the leaves, trunks and roots of the plants enters the soil surface with the withering and falling of these parts; \textcircled{2} (C_{dh}) means that the carbon distributed in the surface layer of soil enters the underground with the humification process; \textcircled{3} (gpp) means that plants (crops) convert inorganic carbon in the air into organic carbon through photosynthesis; \textcircled{4} (Cₜᵣᵣ) means that plants emit carbon into the air in the form of carbon dioxide through respiration; \textcircled{5} (N_{f}) means that the organic nitrogen distributed in the leaves, trunks and roots of plants will wither and fall into the soil surface with these parts; \textcircled{6} (N_{dh}) represents that nitrogen distributed in the surface layer of soil enters the underground with the humification process; \textcircled{7} stands for the nitrogen of the precipitation settlement; \textcircled{8} (N fix) means that the nitrogen in the air is transformed into organic nitrogen in the plants (crops) through biological nitrogen fixation;

where, C_{f} stands for a flux of litter-fall from leaf, trunk, and root as in carbon; C_{dh} a flux of detritus humification as in carbon; gpp stands for a flux of photosynthesis as in gross primary production; Cₜᵣᵣ stands for a flux of respiration of trunk and root; N_{f} stands for a flux of litter-fall from leaf, trunk, and root as in nitrogen; N_{dh} stands for a flux of detritus humification as in nitrogen; N_{fix} stands for a flux of nitrogen fixation as in nitrogen.

Further, in the interrelation of nitrogen variables, the nitrogen pollution load calculation model is involved, and the nitrogen pollution load calculation model consists of calculation of nitrogen settlement from precipitation sources, calculation of nitrogen absorption by plants (crops), calculation of denitrification of nitrate nitrogen and calculation of nitrogen leaching, as follows:

1) Nitrogen settlement from precipitation sources:

Nitrogen settlement from precipitation refers to the process that nitrogen enters the agricultural ecosystem from the atmosphere through precipitation. Due to air pollution and atmospheric circulation, precipitation often contains ammonia and nitrate nitrogen, which settles to the surface with the precipitation settlement. The calculation formula of ammonia and nitrate nitrogen content with precipitation settlement is as follows:

depo_{AMM}=⅓=C_{N}×N_{PRE} 3.7

Similarly, the calculation formula of nitrate nitrogen content with precipitation settlement is as follows:

depo_{NIT}=⅔=C_{N}×N_{PRE} 3.8

where, depo_{AMM} stands for a flux of the ammonia nitrogen in the atmospheric settlement, depo_{NIT} stands for a flux of the nitrate nitrogen in the atmospheric settlement, C_{N} stands for a flux of the nitrogen settlement coefficient, and N_{PRE} stands for a flux of the nitrogen element in the precipitation.

2) The calculation formulae of nitrogen element absorption by the plants (crops) is as follows:

$${N}_{uptake} = ( \frac{{N}_{max} {K}_{s , update} ( {N}_{AMM} + {N}_{NIT} )}{{K}_{uptake} + {K}_{s , uptake} ( {N}_{AMM} + {N}_{NIT} )} ) \times {Q}_{10 , {N}_{uptake}}^{\frac{{T}_{S} - {T}_{opt , uptake}}{10}} \\ {K}_{s , uptake} = 0.9 \times {SW}_{{I}^{3}} + 0.1 ; \qquad 3.9$$

where, Nₘₐₓ represents a flux of the maximum nitrogen uptake of the crops, N_{AMM} represents a flux of the ammonia nitrogen content in the soil, N_{NIT} represents a flux of the nitrate nitrogen content in the soil, and SWI represents a flux of the soil moisture index.

3) The deoxidation calculation formulae of nitrate nitrogen is as follows:

$$denitr = {N}_{NIT} [ 1 - exp ( - 1.4 {f}_{deni , t} C ) ] {num}_{day} \\ {f}_{deni , t} = max [ \begin{matrix}0.1 \\ \frac{t}{t + exp ( 9.93 - 0.312 t )}\end{matrix} ] ; \qquad 3.1$$

where, denitr stands for a flux of denitrification of the nitrate nitrogen, N_{NIT} stands for a flux of nitrate nitrogen content in the soil, and num_{day} stands for a flux of a number of days;

4) Leaching of the nitrate nitrogen:

Nitrogen leaching refers to the nitrogen loss caused by nitrogen in the soil migrating below the active layer of root system with water, but not being absorbed and utilized by crops. Leaching nitrogen mainly includes soil nitrogen and residual fertilizer nitrogen, and the main form of nitrogen leaching is nitrate nitrogen, and the calculation formula is as follows:

$$λ = \frac{1}{b} ;$$

where, Kₛ is a flux of the unsaturated permeability coefficient of the soil, SWI is a flux of the soil moisture index, and λ is a flux of the pore size distribution index;

the formula for calculating the Dore size distribution index is as follows:

$$K ( SWI ) = {K}_{3} {SWI}^{3 + \frac{2}{λ}} ; \qquad 3.11$$

where b is a flux of an empirical constant.

Because the soil texture of each grid is different, the corresponding unsaturated permeability coefficient Kₛ and the empirical constant b are different. In the calculation process, different grids may refer to Table 1.1 for parameter selection:

TABLE 1.1
1 Permeability coefficient and empirical
constant under different soil textures

| NO. | Soil texture | Kₛ | b | Soil types |
|---|---|---|---|---|
| 1 | Coarse sand | 1.41 × 10⁻⁵ | 4.26 | Loamy sand |
| 2 | Coarse powder/sand | 5.23 × 10⁻⁶ | 4.74 | Sandy loam |
| 3 | Coarse powder | 3.38 × 10⁻⁶ | 5.25 | Loam |
| 4 | Clay particle/ | 4.45 × 10⁻⁶ | 6.77 | Sandy clay |
| | Coarse powder | | | loam |
| 5 | Clay particle | 2.45 × 10⁻⁶ | 8.17 | Clay loam |
| 6 | Frozen earth | — | — | — |
| 7 | Organic soil | 3.38 × 10⁻⁶ | 5.25 | Loam |
| 0 | Seawater soil | — | — | — |

Through steps S1021 and S1022, the nitrogen pollution biogeochemical circulation model is obtained.

This embodiment completes the definition of the nitrogen pollution biogeochemical circulation model in FIG. 2 through the definition of six important variables in plants (crops), soil and air, as well as the description of the calculation methods and the interrelations of the variables, and calculates the nitrogen pollution on the surface and underground respectively through this model, which provides reasonable and reliable input data for the calculation of nitrogen pollution in the watershed.

S103, the nitrogen pollution biogeochemical circulation model is established in each of the watershed grids; and

S104, a total non-point source nitrogen pollution in the watershed is calculated according to the nitrogen pollution biogeochemical circulation model in each of the watershed grids.

It should be noted that although the method operations of the above embodiments are described in the drawings in a specific order, this does not require or imply that these operations must be performed in this specific order, or that all the illustrated operations must be performed to achieve the desired results. On the contrary, the depicted steps may change the execution order. Additionally or alternatively, some steps may be omitted, multiple steps may be combined into one step for execution, and/or one step may be decomposed into multiple steps for execution.

### Embodiment 2

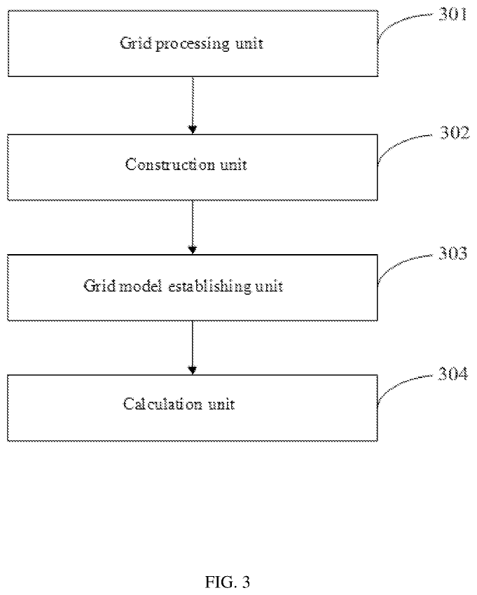

As shown in FIG. 3, this embodiment provides a system for nitrogen pollution hierarchical calculation in a water environment, which includes a grid processing unit 301, a construction unit 302, a grid model establishing unit 303, and a calculation unit 304. The specific functions of each unit are as follows:

a grid processing unit 301 is used for carrying out a gridding processing on a watershed to obtain watershed grids;

a construction unit 302 is used for constructing a nitrogen pollution biogeochemical circulation model according to calculation methods and interrelations of t carbon and nitrogen variables;

a grid model establishing unit 303 for constructing the nitrogen pollution biogeochemical circulation model in each of the watershed grids; and

a calculation unit 304 is used for calculating the total non-point source nitrogen pollution in the watershed according to the nitrogen pollution biogeochemical circulation model in each of the watershed grids.

The specific implementation of each unit in this embodiment may be found in the above-mentioned Embodiment 1, and will not be described here. It should be noted that the system provided by this embodiment only illustrates the division of the above-mentioned functional units. In practical application, the above-mentioned functional allocation may be completed by different functional units as needed, that is, the internal structure may be divided into different functional units to complete all or part of the above-mentioned functions.

### Embodiment 3

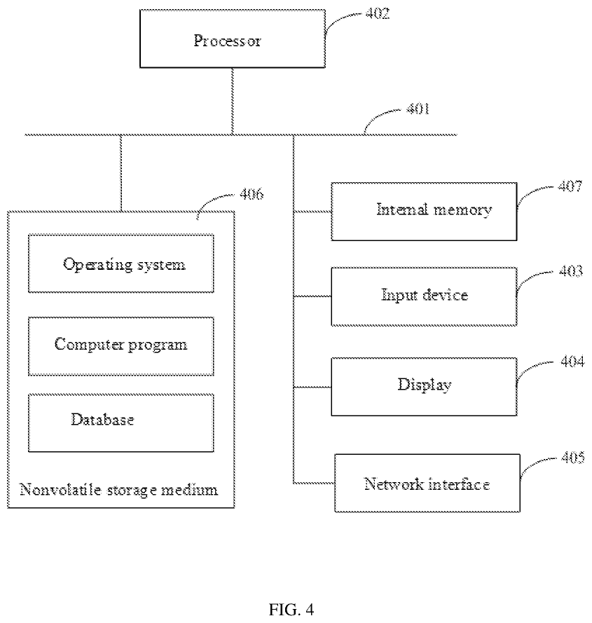

As shown in FIG. 4, this embodiment provides a computer equipment, which includes a processor 402, a memory, an input device 403, a display device 404 and a network 405 connected through a system bus 401. The processor is used to provide computing and control capabilities, and the memory includes a nonvolatile storage medium 406 and an internal memory 407. The nonvolatile storage medium 406 stores an operating system, a computer program and a database. The internal memory 407 provides an environment for the operation of the operating system and computer program in the nonvolatile storage medium. When the processor 402 executes the computer programs stored in the memory, the method for nitrogen pollution hierarchical calculation in a water environment in Embodiment 1 above is realized as follows:

a gridding processing is carried out on a watershed to obtain watershed grids;

a nitrogen pollution biogeochemical circulation model is constructed according to calculation methods and interrelations of carbon and nitrogen variables;

the nitrogen pollution biogeochemical circulation model is established in each of the watershed grids; and

a total non-point source nitrogen pollution in the watershed is calculated according to the nitrogen pollution biogeochemical circulation model in each of the watershed grids.

### Embodiment 4

This embodiment provides a storage medium, which is a computer-readable storage medium for storing computer programs. When the computer programs are executed by a processor, the method for nitrogen pollution hierarchical calculation in a water environment in Embodiment 1 above is realized as follows:

a gridding processing is carried out on a watershed to obtain watershed grids;

a nitrogen pollution biogeochemical circulation model is constructed according to calculation methods and interrelations of carbon and nitrogen variables;

the nitrogen pollution biogeochemical circulation model is established in each of the watershed grids; and

a total non-point source nitrogen pollution in the watershed is calculated according to the nitrogen pollution biogeochemical circulation model in each of the watershed grids.

It should be noted that the computer-readable storage medium of this embodiment may be a computer-readable signal medium or a computer-readable storage medium or any combination of the two. The computer-readable storage medium may be, for example, but not limited to, an electrical, magnetic, optical, electromagnetic, infrared, or semiconductor system, device or apparatus, or any combination of the above. More specific examples of computer-readable storage media may include, but are not limited to, an electrical connection with one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage apparatus, a magnetic storage apparatus, or any suitable combination of the above.

In this embodiment, the computer-readable storage medium may be any tangible medium containing or storing a program, and the program may be used by or in combination with an instruction execution system, device or device. In this embodiment, the computer-readable signal medium may include a data signal propagated in baseband or as a part of a carrier wave, in which a computer-readable program is carried. This propagated data signal may take many forms, including but not limited to electromagnetic signals, optical signals or any suitable combination of the above. The computer-readable signal medium may also be any computer-readable storage medium other than the computer-readable storage medium, which may send, propagate or transmit the program for use by or in connection with the instruction execution system, device or apparatus. The computer program contained in the computer-readable storage medium may be transmitted by any suitable medium, including but not limited to: wires, optical cables, RF (radio frequency) and the like, or any suitable combination of the above.

The computer-readable storage medium may be used to write a computer program for executing this embodiment in one or more programming languages or their combinations, including object-oriented programming languages, such as Java, Python and C++, and conventional procedural programming languages, such as C language or similar programming languages. The program may be executed entirely on the user's computer, partially on the user's computer, as an independent software package, partially on the user's computer and partially on a remote computer, or completely on a remote computer or server. In the case involving a remote computer, the remote computer may be connected to a user computer through any kind of network, including a local area network (LAN) or a wide area network (WAN), or may be connected to an external computer (for example, Internet connection using an Internet service provider).

To sum up, the application specifically describes the circulation of nitrogen among plants, soil and humus soil, so that the nitrogen circulation process in different grids may be more accurately presented, and the scientificity and reliability are greatly improved compared with the previous methods; moreover, the description of different forms of nitrogen elements in each space may reflect the spatial and temporal distribution of non-point source pollution nitrogen elements, so that the pollution evaluation result is more comprehensive.

The above is only the preferred embodiment of the patent of the present application, but the scope of protection of the patent of the present application is not limited to this. Any person familiar with the technical field within the scope disclosed in the patent of the present application may replace or change it equally according to the technical scheme and inventive concept of the patent of the present application, which belongs to the scope of protection of the patent of the present application.

What is claimed is:

1. A method for nitrogen pollution hierarchical calculation in a water system, comprising following steps:
carrying out a gridding processing on a watershed to obtain watershed grids;
constructing a nitrogen pollution biogeochemical circulation model according to calculation methods and interrelations of carbon and nitrogen variables, wherein constructing the nitrogen pollution biogeochemical calculation model comprises:
determining a variable C_{VEG} of an organic carbon element content in plants, a variable N_{VEG} of an organic nitrogen element content in the plants, a variable C_{DET} of an organic carbon content in a surface soil, a variable N_{DET} of an organic nitrogen content in the surface soil, a variable N_{HUM} of the organic carbon content in a humus soil and a variable N_{HUM} of the organic nitrogen content in the humus soil; and
determining the calculation methods and the interrelations of C_{VEG}, N_{VEG}, C_{DET}, N_{DET}, C_{HUM}and N_{HUM}, and completing a construction of the nitrogen pollution biogeochemical circulation model according to a nitrogen pollution load calculation model;
wherein the calculation methods of C_{VEG}, N_{VEG}, C_{DET}, N_{DET}, C_{HUM} and N_{HUM} are as follows:
a calculation method of the organic carbon content in the plants,
$$\frac{\partial {C}_{VEG}}{\partial t} = gpp - {C}_{trr} - {C}_{f} ;$$
a calculation method of the organic carbon content in the surface soil,
$$\frac{\partial {C}_{DET}}{\partial t} = {C}_{f} - {C}_{dr} - {C}_{dh} ;$$
a calculation method of the organic carbon content in the humus soil,
$$\frac{\partial {C}_{HUM}}{\partial t} = {C}_{dh} - {C}_{hr} - {C}_{hcar} ;$$
a calculation method of the organic nitrogen content in the plants,
$$\frac{\partial {N}_{VEG}}{\partial t} = {N}_{uptake} - {N}_{f} + {N}_{fix} ;$$
a calculation method of the organic nitrogen content in the surface soil,
$$\frac{\partial {N}_{DET}}{\partial t} = {N}_{f} - {N}_{mind} - {N}_{dh} ;$$
a calculation method of the organic nitrogen content in the humus soil,
$$\frac{\partial {N}_{HUM}}{\partial t} = {N}_{dh} - {N}_{min h} ;$$
wherein stands for a carbon assimilation rate of a photosynthesis assimilation of the plants, Cₜᵣᵣ stands for a carbon emission rate released by a respiration of the plants, C_{f} stands for a carbon cycle rate of the plants, C_{dr} stands for a carbon element decomposed by an organic matter, C_{dh} stands for a carbon humification of the organic matter, Cₕᵣ stands for a carbon element decomposition in the humus soil, Cₕₑₐᵣ stands for a carbonization of the carbon element, Nᵤₚₜₐₖₑ stands for inorganic nitrogen absorbed by the plants, N_{f} stands for a nitrogen cycle rate of the plants, N_{fix} stands for a nitrogen fixation rate, N_{mind} stands for a mineralization of the organic nitrogen, N_{dh} stands for a nitrogen humidification of the organic matter, and N_{mind} stands for an inorganic nitridation of the humus soil;
constricting the nitrogen pollution biogeochemical circulation model in each of the watershed grids; and
calculating a total non-point source nitrogen pollution in the watershed according to the nitrogen pollution biogeochemical circulation model in each of the watershed grids.

2. (canceled)

3. (canceled)

4. The method for nitrogen pollution hierarchical calculation in a water system according to claim 1, wherein the nitrogen pollution load calculation model comprises a calculation of a nitrogen pollution settlement from precipitation sources, a calculation of a nitrogen element absorption by crops, a calculation of a nitrate nitrogen deoxidation process, and a calculation of a nitrate nitrogen leaching.

5. The method for nitrogen pollution hierarchical calculation in a water system according to claim 4, wherein a nitrogen pollution from precipitation sources comprises an ammonia nitrogen element by a precipitation settlement and nitrate nitrogen element by the precipitation settlement; and a calculation formula of the ammonia nitrogen element by the precipitation settlement is as follows:
depo_{AMM}=⅓×C_{N}×N_{PRE},
a calculation formula of the nitrate nitrogen element by the precipitation settlement is as follows:
depo_{NIT}=⅔×C_{N}×N_{PRE},
wherein depo_{AMM} stands for the ammonia nitrogen in an atmospheric settlement, depo_{NIT} stands for nitrate nitrogen in the atmospheric settlement, C_{N} stands for a nitrogen settlement coefficient, and N_{PRE} stands for a nitrogen element in a precipitation.

6. The method for a nitrogen pollution hierarchical calculation in a water system according to claim 4, wherein specific formulae for calculating the nitrogen absorption by the crops are as follows:
$${N}_{uptake} = ( \frac{{N}_{max} {K}_{s , update} ( {N}_{AMMM} + {N}_{NIT} )}{{K}_{uptake} + {K}_{s , uptake} ( {N}_{AMM} + {N}_{NIT} )} ) \times {Q}_{10 , {N}_{uptake}}^{\frac{{T}_{s} - {T}_{opt , uptake}}{10}} \\ {K}_{s , uptake} = 0.9 \times {SWI}^{3} + 0.1 ;$$
wherein Nₘₐₓ stands for a maximum nitrogen uptake of the crops, N_{AMM} stands for an ammonia nitrogen content in the soil, N_{NIT} stands for a nitrate nitrogen content in soil, and SWI stands for a soil moisture index.

7. The method for nitrogen pollution hierarchical calculation in a water system according to claim 4, wherein specific formulae for calculating the nitrate nitrogen deoxidation process and the nitrate nitrogen leaching are as follows:
the calculation of the nitrate nitrogen deoxidation process:
$$denitr = {N}_{NIT} [ 1 - exp ( - 1.4 {f}_{deni , t} C ) ] {num}_{day} \\ {f}_{deni , t} = max [ \begin{matrix}0.1 \\ \frac{t}{t + exp ( 9.93 - 0.312 t )}\end{matrix} ] ;$$
wherein denitr stands for a denitrification of the nitrate nitrogen, N_{NIT} stands for the nitrate nitrogen content in the soil, and num_{day} stands for a number of days;
the calculation of the nitrate nitrogen leaching:
$$K ( SWI ) = {K}_{s} {SWI}^{3 + \frac{2}{λ}} ;$$
wherein Kₛ is a soil unsaturated permeability coefficient, SWI is the soil moisture index and λ is a pore size distribution index;
the formula for the pore size distribution index is as follows:
$$λ = \frac{1}{b} ;$$
wherein b is an empirical constant.

8. A system for nitrogen pollution hierarchical calculation in a water system comprising:
a grid processing unit used for carrying out a gridding processing on a watershed to obtain watershed grids;
and a construction unit used for constructing a nitrogen pollution biogeochemical circulation model according to calculation methods and interrelations of carbon and nitrogen variables; wherein constructing the nitrogen pollution biogeochemical circulation model comprises:
determining a variable C_{VEG} of an organic carbon element content in plants, a variable N_{VEG} of an organic nitrogen element content in the plants, a variable C_{DET} of an organic carbon content in a surface soil, a variable N_{DET} of an organic nitrogen content in the surface soil, a variable C_{HUM} of the organic carbon content in a humus soil and a variable N_{HUM} of the organic nitrogen content in the humus soil; and
determining the calculation methods and the interrelations of C_{VEG}, N_{VEG}, C_{DET}, N_{DET}, C_{HUM}and N_{HUM}, and completing a construction of the nitrogen pollution biogeochemical circulation model according to a nitrogen pollution load calculation model;
wherein the calculation methods of C_{VEG}, N_{VEG}, C_{DET}, N_{DET}, C_{HUM} and N_{HUM} are as follows:
a calculation method of the organic carbon content in the plants;
$$\frac{\partial {C}_{VEG}}{\partial t} = gpp - {C}_{trr} - {C}_{f}$$
a calculation method of the organic carbon content in the surface soil;
$$\frac{\partial {C}_{DET}}{\partial t} = {C}_{f} - {C}_{dr} - {C}_{dh}$$
a calculation method of the organic carbon content in the humus soil;
$$\frac{\partial {C}_{HUM}}{\partial t} = {C}_{dh} - {C}_{hr} - {C}_{hcar}$$
a calculation method of the organic nitrogen content in the plants;
$$\frac{\partial {N}_{VEG}}{\partial t} = {N}_{uptake} - {N}_{f} + {N}_{fix}$$
a calculation method of the organic nitrogen content in the surface soil;
$$\frac{\partial {N}_{DET}}{\partial t} = {N}_{f} - {N}_{mind} - {N}_{dh}$$
a calculation method of the organic nitrogen content in the humus soil;
$$\frac{\partial {N}_{HUM}}{\partial t} = {N}_{dh} - {N}_{min h}$$
wherein gpp stands for a carbon assimilation rate of a photosynthesis assimilation of the plants, Cₜᵣᵣ stands for a carbon emission rate released by a respiration of the plants, C_{f} stands for a carbon cycle rate of the plants, C_{dr} stands for a carbon element decomposed by an organic matter, C_{dh} stands for a carbon humification of the organic matter, Cₕᵣ stands for a carbon element decomposition in the humus soil, Cₕₑₐᵣ stands for a carbonization of the carbon element, Nᵤₚₜₐₖₑ stands for inorganic nitrogen absorbed by the plant, N_{f} stands for a nitrogen cycle rate of the plants, N_{fix} stands for a nitrogen fixation rate, N_{mind} stands for a mineralization of the organic nitrogen N_{dh} stands for a nitrogen humidification of the organic matter, and N_{min h} stands for an inorganic nitridation of the humus soil;
a grid model constructing unit, used for constructing the nitrogen pollution biogeochemical circulation model in each of the watershed grids; and
a calculation unit, used for calculating a total non-point source nitrogen pollution in the biogeochemical circulation model in each of the watershed grids; and
a calculation unit, used for calculating total non-point source nitrogen pollution in the watershed according to the nitrogen pollution biogeochemical circulation model in each of the watershed grids.

9. A computer equipment, comprising a processor and a memory for storing programs executable by the processor, wherein when the processor executes the programs stored in the memory, the method for nitrogen pollution hierarchical calculation in a water system according to claim 1 is realized.
